(19) United States
(12) Patent Application Publication
(10) Pub. No.: US 2011/0046517 A1
(43) Pub. Date: Feb. 24, 2011

(54) METHOD AND SYSTEM FOR MONITORING SKELETAL DEFECTS

(76) Inventor: Sanford A. Roth, New York, NY (US)

Correspondence Address: PORZIO, BROMBERG & NEWMAN,P.C., Suite 201, 29 Thanet Road, Princeton, NJ, 08540, US

(21) Appl. No.: 12/781,136

(22) Filed: May 17, 2010

Related U.S. Application Data
(60) Provisional application No. 61/178,520, filed on May 15, 2009.

(51) Int. Cl. A61B 5/103

(52) U.S. Cl. 600/587

(57) ABSTRACT

The present invention relates to a handheld, point-of-care device that uses non-hazardous low level ultrasound to quantitatively detect, monitor and supply, in real time, information on the status of bone fractures from inception to full healing. The device includes a plurality of transducers housed in the handheld unit or which can be extended from the handheld unit to be placed at or near the site of the bone fracture. A plurality of acoustic parameters of the transducers are selected to provide optimal detection and monitoring of fractures in bone. The information can be displayed as a numerical readout indicating the severity of the break and provides ability for indicating small stress and fatigue fractures. The portable device provides rapid and inexpensive detection and diagnosis of musculoskeletal problems using low level ultrasound which can measure bone density, determine fracture status and monitor healing rate.

### CROSS REFERENCE TO RELATED APPLICATION

This application claims the benefit of U.S. Provisional Patent Application No. 61/178,520, filed May 15, 2009, the entirety of which is hereby incorporated by reference into this application.

### BACKGROUND OF THE INVENTION

Current methods of detection including x-rays, MRIs and CTs for detecting bone fractures are qualitative, subjective, and costly. They require large expensive equipment, specialized facilities, reading and interpretation by trained personnel and safety precautions.

U.S. Pat. No. 5,143,069 describes a diagnostic method of monitoring skeletal defect by in vivo acoustical measurement of mechanical strength using correlation and spectral analysis. A pair of transducers are mounted over the skin and an ultrasound signal is propagated from one of the transducers along the hard tissues and surrounding soft tissues. The propagated signal is received at the other transducer. The mechanical strength of the hard tissues is determined on the basis of the ultrasound parameters, including the amount of energy propagated, the velocity of the ultrasound and the degree of dispersion together with the characteristic response of the hard tissues. The transducer is wide band type having a bandwidth greater than 100 KHz and an approximate resonance frequency of 1 MHz.

It is desirable to provide an improved device which is capable of detecting fatigue and stress fractures providing for earlier and more precise intervention.

### SUMMARY OF THE INVENTION

The present invention relates to a handheld, point-of-care device that uses non-hazardous low level ultrasound to quantitatively detect, monitor and supply, in real time, information on the status of bone fractures from inception to full healing. The device includes a plurality of transducers housed in the handheld unit or which can be extended from the handheld unit to be placed at or near the site of the bone fracture. A plurality of acoustic parameters of the transducers are selected to provide optimal detection and monitoring of fractures in bone. The information can be displayed as a numerical readout indicating the severity of the break and provides ability for indicating small stress and fatigue fractures. The portable device provides rapid and inexpensive detection and diagnosis of musculoskeletal problems using low level ultrasound which can measure bone density, determine fracture status and monitor healing rate.

The device provides consistent, quantitative measurements, minimizing interpretive error. The data provides high accuracy, stability and sensitivity for indication of fracture status. The device of the present invention can supply medical personnel with data that provides high accuracy, stability and sensitivity to conveniently evaluate and optimally treat injuries. The selected acoustic parameters and measurements can be stored and the same parameters can later be used to collect additional data. Using the stored data, the device can be used to develop a record of bone strength measurements. The device provides measurement and analysis of: bone fractures and microfractures; healing rate; identification of non-unions and hairline fractures; osteoporosis and prediction of bone abnormalities. The device of the present invention has the following advantages: it is a non-invasive, lightweight and portable device that can be used at the point of care; can be easily used by paramedical personnel; can give real-time measurements; can give quantitative support for the physician's diagnosis; and can measure fracture status when implants and fixation devices are used.

The invention will be more fully described by reference to the following drawings.

### BRIEF DESCRIPTION OF THE DRAWINGS

FIG. 1 is a schematic bottom plan view of a handheld portable ultrasound diagnostic device in accordance with the teachings of the present invention.

FIG. 2 is schematic side plan view of handheld portable ultrasound diagnostic device.

FIG. 3 is a representation of a bovine testing device.

FIG. 4 is a schematic diagram of acoustic parameters.

FIG. 5 is a schematic diagram of a configuration of system for gathering and evaluating data from the handheld portable ultrasound diagnostic device.

FIG. 6 is a schematic block diagram for signal conditioning, correlation analysis and spectral estimation of the present invention.

FIG. 7 is a schematic diagram of a normal healing display screen.

FIG. 8 is a schematic diagram of a delayed healing display screen.

FIG. 9A is a graph of signal amplitude.

FIG. 9B is a graph of energy transmitted.

FIG. 9C is a graph of flight time.

FIG. 9D is a graph of pulse duration.

FIG. 9E is a graph of the number of counts over a predetermined threshold.

### DETAILED DESCRIPTION

Reference will now be made in greater detail to a preferred embodiment of the invention, an example of which is illustrated in the accompanying drawings. Wherever possible, the same reference numerals will be used throughout the drawings and the description to refer to the same or like parts.

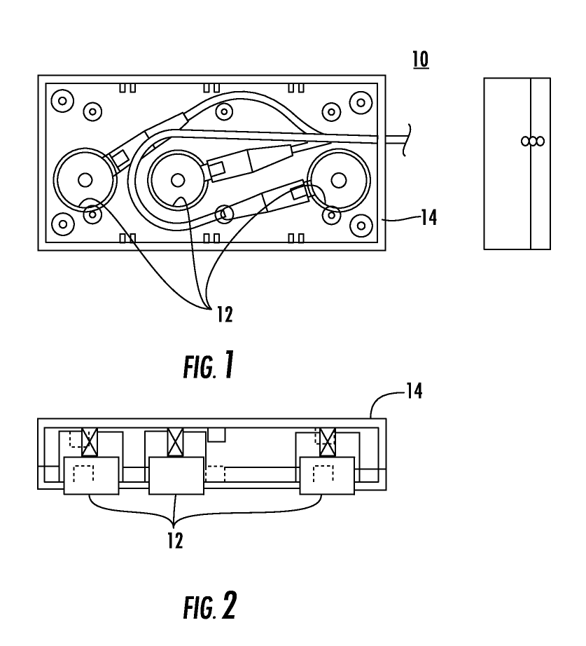

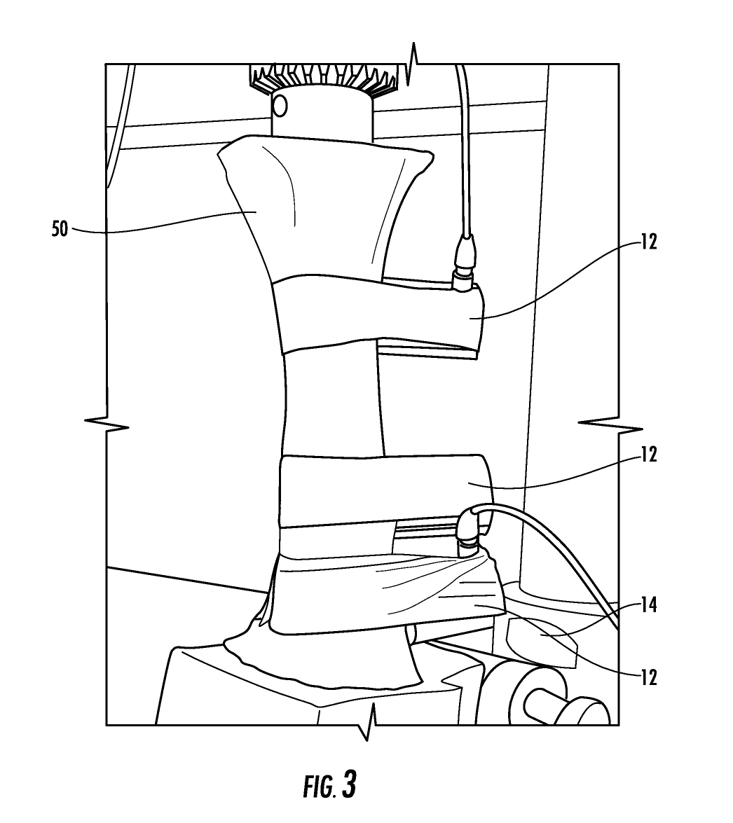

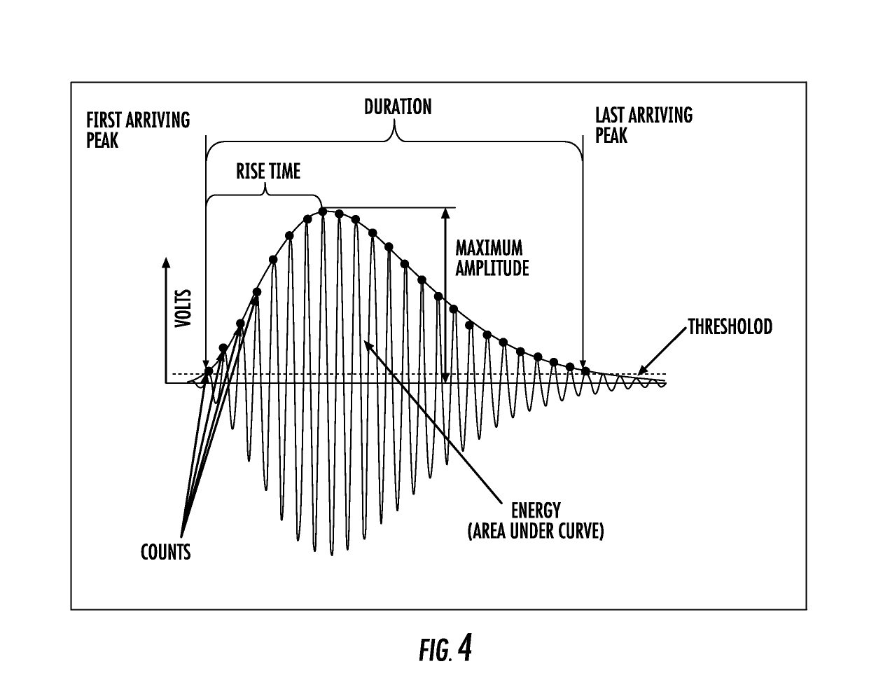

FIGS. 1 and 2 are schematic diagrams of handheld portable ultrasound diagnostic device 10 in accordance with the teachings of the present invention. As an example, FIG. 3 is a schematic diagram of representative device 10 including transducer 12 upon application to soft tissue 50. Transducers 12 are contained within housing 14. In one embodiment, transducers 12 can extend from housing 14. Housing 14 and/or transducers 12 can be placed at or near a site of a bone fracture. Transducers 12 can be focused and angled to propagate a signal through the soft tissue and along the bone. The angle of transducers 12 can vary as a function of soft tissue thickness. Transducers 12 can have a resonance frequency in the range of 300 KHz to 2 MHz. Preferably, the resonance frequency of transducers 12 is greater than 1 MHz.

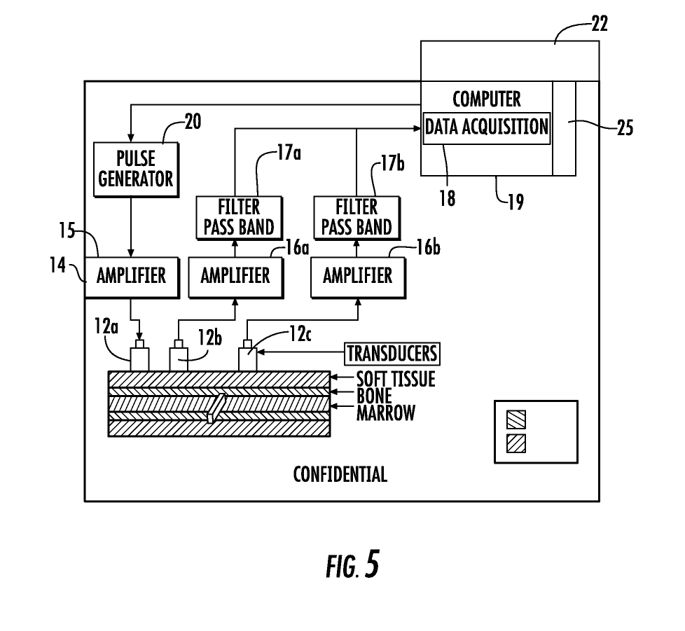

A plurality of acoustic parameters from transducers 12 are selected in a way to provide optimal detection and monitoring of fractures in bone. Example acoustic parameters are shown in FIG. 4. Acoustic parameters can include flight time, as the time it takes for acoustical energy to travel along a test specimen from transmitting transducer to receiving transducer. The first arriving peak is determined from the first peak that exceeds a sensitivity threshold. Other acoustic parameters that can be selected to provide detection and monitoring of fractures in bone include the duration, rise time, maximum amplitude, counts and energy. Duration is the time between the first arriving peak and the last arriving peak. Rise time is the time between the first arriving peak and the peak with the maximum amplitude. Maximum amplitude is the voltage of the highest peak. Counts are a measure of the number of peaks arriving over the course of the duration of the energy packet. The final measure is the amount of energy in the packet. This is calculated as the area under the curve. It has been found that there is a direct correlation between the speed and sound along bone and density of the bone. The measurement of the bone density can be used to determine the fracture status and monitor the healing rate. Acoustic measurements can also determine a velocity, propagation energy, and degree dispersion of the ultrasound signal propagated along the bone and the surrounding soft tissue based upon the propagated ultrasound signal and the velocity, propagated energy and degree of dispersion can be related to the mechanical strength and the structural integrity of the bone as described in U.S. Pat. No. 5,143,069 hereby incorporated by reference in its entirety into this application.

FIG. 5 is a schematic diagram of a test configuration of system 100 for gathering and evaluating data from the handheld portable ultrasound diagnostic device 10. Transducers 12 are excited by pulse generator 20 applying a signal through amplifier 15 to transducer 12a. In one embodiment, transducer 12a can be used as a transmitter and transducers 12b-12c can be used as receivers. Transducers 12a-12c can be mounted to soft tissue 22 at or near bone fracture 24. Amplifiers 16a,16b and filters 17a,17b condition the received signals which are forwarded to data acquisition module 18 of processor 19. Processor 19 conducts the selection of acoustical parameters and signal processing procedures. Processor 19 can be, for example, a CPU general purpose processor or integrated circuit which under normal operation processes data under the control of an operating system and application software stored in Random Access Memory and/or Read Only Memory. Display 22 can display in real-time at housing 14, processed data. For example, display 22 can be a liquid crystal display. Data of acoustical parameters and measurements can be stored in memory 25.

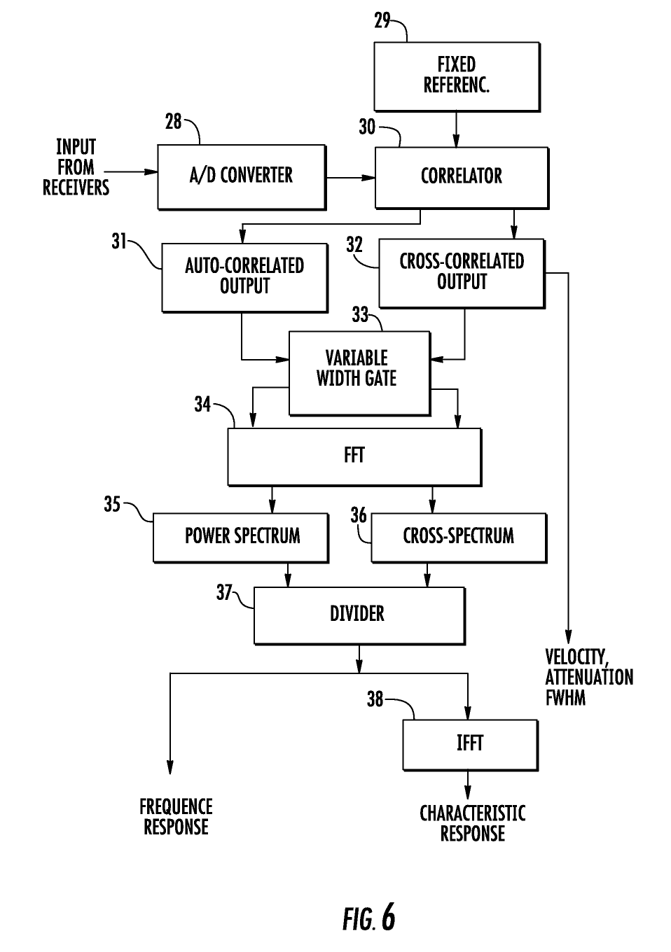

As shown in FIG. 6, an analog-to-digital converter 28 digitizes received information from device 10. A fixed reference signal 29 is generated by joining the transmitter and one of the receivers face-to-face and is stored in a memory (not shown). A digital correlator 30 calculates an auto-correlation 31 of the received ultrasound signals and a cross-correlation 32 of the received ultrasound signal using the fixed reference signal. The correlated signals are applied to the computer 34 and displayed on a display 22, as shown in FIG. 5.

The mechanical strength and structural integrity of hard tissues, such as bone, can be determined by analyzing any correlated signal in terms of ultrasound parameters including the velocity of the ultrasound in the tissue, attenuation and the degree of dispersion of the ultrasound signal while propagating through the tissue. A variable delay gate 33, having a starting position and a width that can be determined by those skilled in art interactively through the monitor 35, limits the range of the correlated output to separate the ultrasound energy propagated along the soft tissues from the ultrasound energy propagated along the hard tissue. The auto-correlated signal and cross-correlated signal can be represented, in the frequency domain, by a fast Fourier transform (FFT) 34 as an approximated power spectrum 35 and cross-spectrum 36, respectively. A digital divider 37 is used to obtain the approximated characteristic frequency response of the bone. The time domain representation of the approximated frequency response of the bone can be obtained through the inverse Fourier transforms (IFFT) 38. The characteristic response of the bone both in the frequency domain and in the time domain can then be used to predict the risk of failure.

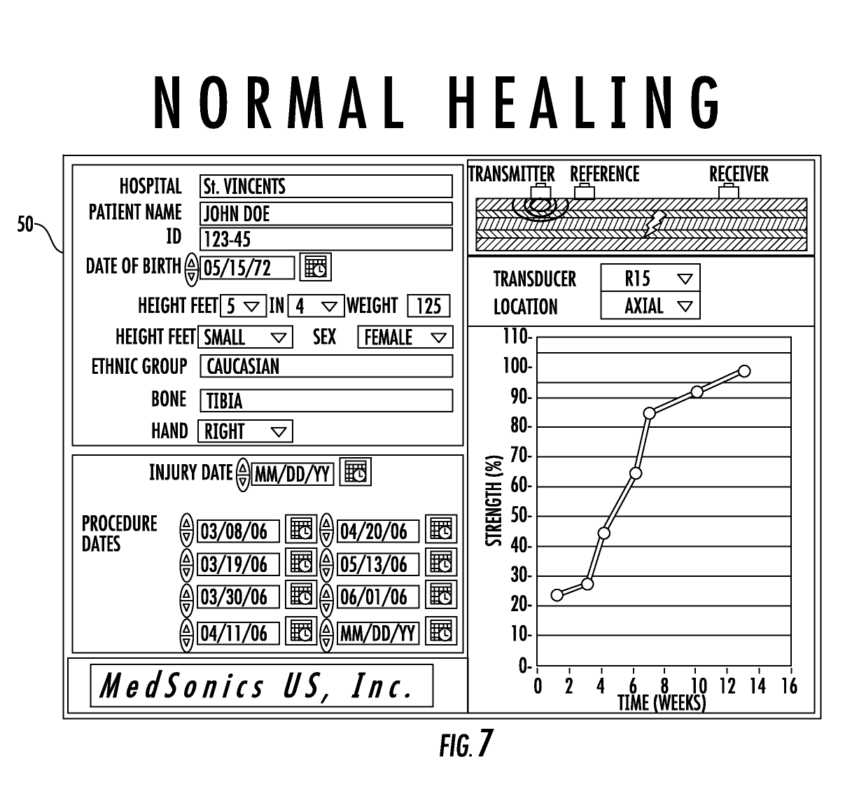

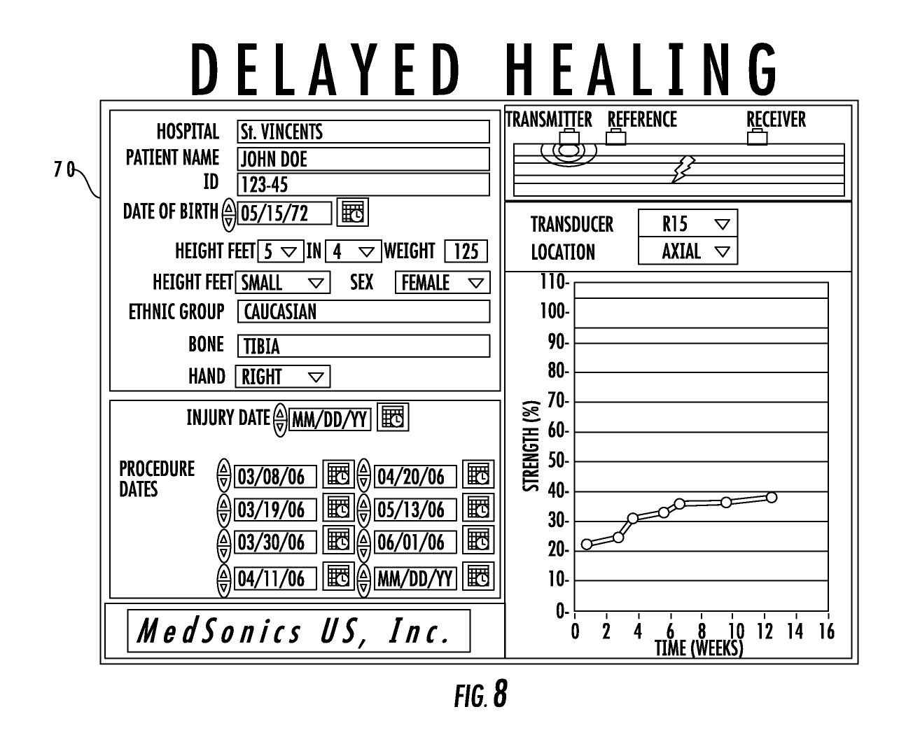

Diagnostic device 10 provides the physician with diagnostic support information of the processed data. An example of normal healing display screen 50 is shown in FIG. 7. The diagnostic support information can be used to chart over time the status of a healing fracture against the norm of population information of age, sex and ethnicity. Information can be supplied directed to fracture identification, normal healing, early detection of a delayed healing and osteoporosis status. An example of delayed healing screen 70 is shown in FIG. 8.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

### EXAMPLES

### Example 1

### Bovine Femur Testing

Tests were performed on bone with precise cuts using a fine saw to mimic fatigue fractures in the bones cortical surface (hard outer layer) of varying depths and lengths using device 10 upon application to soft tissues as shown in FIG. 3. Five parameters of axial transmission were evaluated to establish the transducer and signal combination which yields the optimal detection and monitoring of fatigue fractures.

### Example 2

### Evaluation and Correlation

Experimental data was gathered for the evaluation of the five parameters of axial transmission, shown in FIG. 4, by using cross correlation techniques which established the detection and monitoring of fatigue fractures. These tests provide comprehensive results for simulated fatigue fractures of various sizes. The results define the combination of transducer frequency and acoustic parameters that have the greatest sensitivity and specificity to the presence of a fracture. The highest sensitivity is defined as the largest change in a parameter's value for the smallest change in fracture size. The highest specificity is defined as the accurate identification and characterization of a fracture. This data provides the critical technical parameters for defining the ultrasonic transducers' characteristics and configuration.

### Example 3

### Test Results

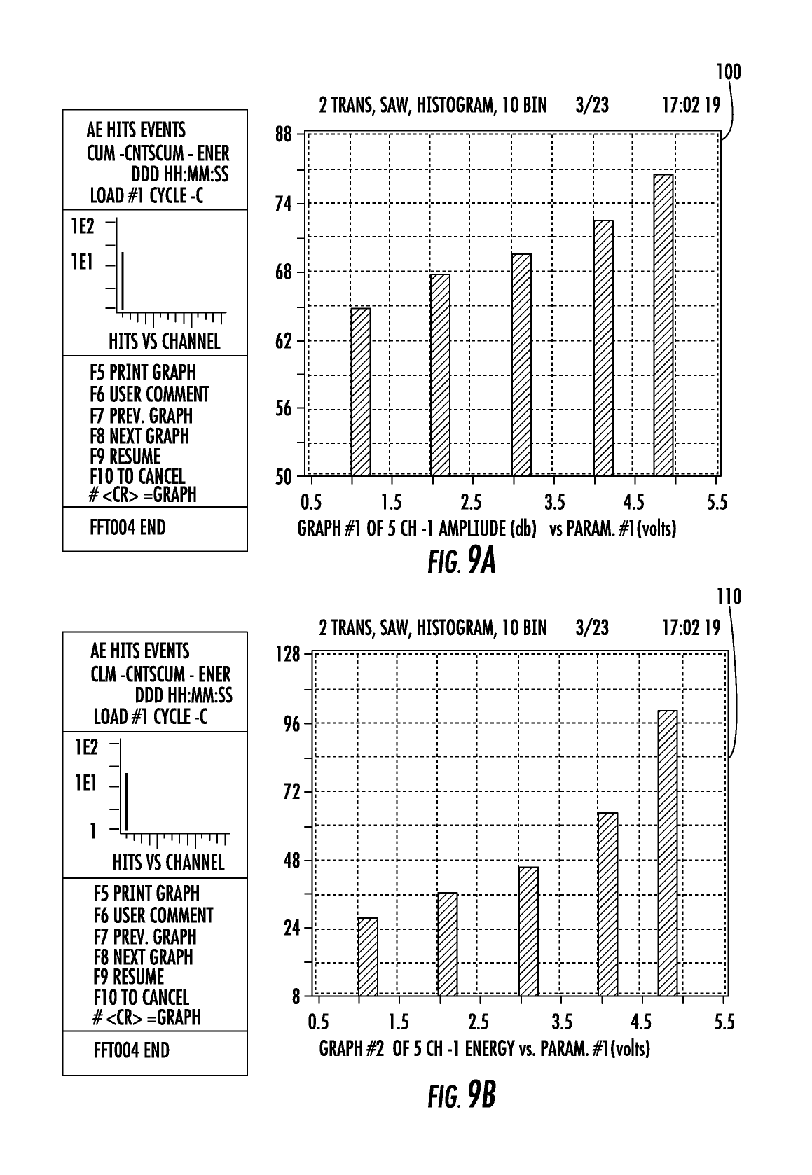

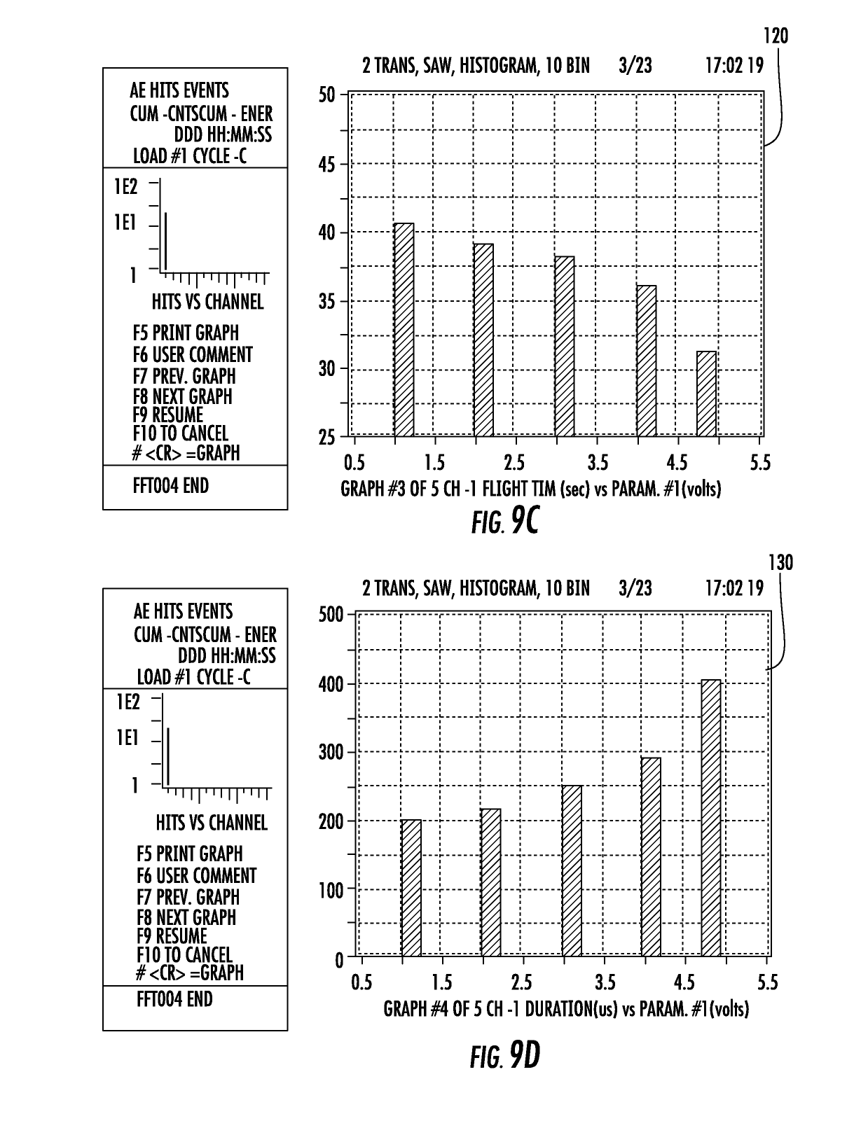

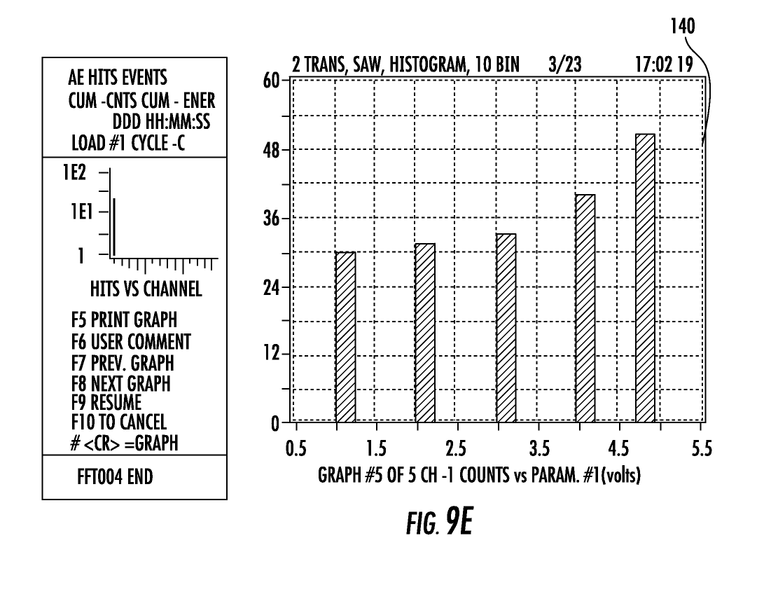

FIG. 9A-9E provides graphs made while performing tests on the bovine femur, as shown in FIG. 3, using system 100, as shown in FIG. 4. Cuts were made by surgically increasing the depth of cut in the cortical portion of the bone and charting the results of the five parameters.

Each graph shows, reading left to right horizontally, the deepest cut on the left of the chart, to the smallest cut and uncut bone on the right of the chart in millimeter depth (5 to 10 mm). As shown, the signal decreases as the depth of the cut increases, on the left of the chart, giving a well defined change as the signal increases with reduction in cut depth at the right of the chart in FIGS. 9A-9E. However, in the case of Flight Time, graph is reversed in FIG. 9C.

These results have shown that system 100 provides improved definition in signal information and thus can indicate a small cut which mimics a stress fracture in all the five parameters measured.

FIG. 9A illustrates graph 100 of signal amplitude. Graph 100 shows the increase in amplitude of the signal as the depth of cut decreases which simulates the healing process. This demonstrates the sensitivity of this parameter to changes in fracture depth and rate of healing.

FIG. 9B illustrates graph 110 of energy transmitted. The change in energy transmitted in graph 110, from deep cut to small cut, indicates the extent of the fracture and healing rate which rises to the normal level in a well defined curve.

FIG. 9C illustrates graph 120 of flight time (time from energy transmission to reception). The reversal to a downward curve in graph 120 from the upward curve in the other graphs is due to the increase in time for the signal to travel from the transmitter to the receiver with an increase in cut depth. With smaller cut depths and normal bone there is a reduction in transmission time between transmitter and receiver.

FIG. 9D illustrates graph 130 of pulse duration (pulse width). Changes in the pulse width, due to changes in cut depth of graph 130, also give a strong indication of changes in fracture extent and healing rate.

FIG. 9E illustrates graph 140 of the number of counts over a predetermined threshold. Graph 140 shows a significant change due to cut depth that allows for maximum sensitivity when there is a change of signal peaks over a predetermined monitoring test level. This shows, as well, the extent of the fracture and healing rate.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed:

1. A method of measuring, in vivo, mechanical strength and structural integrity of bone structure in a body having soft tissue and skin by the following procedures:
(a) mounting three ultrasound transducers over the skin and propagating a signal from one of the transducers, said transducer being focused and angled such that the signal will propagate through the soft tissue and along the bone;
(b) establishing a fixed reference signal for the ultrasound transducers;
(c) receiving the propagated ultrasound signal at another one of said transducers;
(d) sorting the received ultrasound signal according to a relative propagation delay time by correlating the received ultrasound signal with the fixed reference signal;
(e) determining at least one acoustical parameter from the ultrasound signal propagated along the bone and the surrounding soft tissue based upon the propagated ultrasound sorting of (d); and
(f) relating the at least one acoustical parameter to the mechanical strength and the structural integrity of the bone.

2. The method according to claim 1 further comprising the step of:
establishing an additional reference signal by monitoring the ultrasound signal propagated through a normal part of the bone.

3. The method of claim 1 wherein the acoustical parameters are selected from the group of flight time, pulse duration, rise time, maximum amplitude, counts and energy.

4. The method of claim 1 wherein the acoustical parameters are selected from a velocity, a propagation energy and a degree dispersion.

5. A method as claimed in claim 1 wherein step (d) comprises the substep of sorting the received propagated ultrasound signal propagated along the bone and the received propagated ultrasound signal along the soft tissue.

6. The method of claim 1 further comprising the steps of:
storing the mechanical strength and structural integrity of the bone;
repeating steps (a) through (f) over time; and
displaying the mechanical strength and structural integrity of the bone over time.

7. The method of claim 1 wherein the angle of at least one of said transducers can vary as a function of soft tissue thickness.

8. The method of claim 1 wherein the transducers have a resonance frequency greater than 1 MHz.

9. A method of measuring mechanical strength and structural integrity of bone in a body having bone and soft tissue said method comprising:
(a) placing a focused and angled ultrasound transmitter and a first ultrasound receiver over the body with a first distance from the ultrasound transmitter therebetween;
(b) placing a second ultrasound receiver over the body at a second distance less than the first distance from the ultrasound transmitter;
(c) transmitting low level ultrasound into the body so as to propagate the transmitted ultrasound through the soft tissue and along the bone;
(d) generating a fixed reference signal by joining the ultrasound transmitted signal with one of the ultrasound receivers;
(e) receiving ultrasound propagated along the bone at the first and the second ultrasound receivers;
(f) auto-correlating the received ultrasound signals and cross-correlating the received ultrasound signals with the fixed referenced signal;
(g) extracting the received ultrasound signal propagated through the bone from the correlated signals; and
(h) obtaining the approximated frequency response and characteristic response of the bone based on the extracted ultrasound signal.

10. The method according to claim 9 wherein step (g) includes gating the received ultrasound signal in the time domain so as to pass only ultrasound signals propagated through bone.

11. The method according to claim 10 wherein step (g) includes a substep of determining the peak and dispersion of the correlated signals.

12. The method according to claim 11 wherein step (h) further includes a substep of performing a fast Fourier transform (FFT) on said correlated signals to obtain an approximated power spectrum and an approximated cross spectrum of said correlated signals and performing division between them to obtain the approximated frequency response.

13. The method according to claim 11 wherein step (h) includes a substep of performing an inverse fast Fourier transform (FFT) on the approximated frequency response to obtain the characteristic response of the bone.

14. The determining method according to claim 9 comprising the steps of:
(a) determining a time for said ultrasound transmitted in step (b) to travel between the ultrasound transmitter, the first ultrasound receiver and the second ultrasound receiver; and
(b) determining the velocity of ultrasound based upon the time delay and the distance between the ultrasound transmitter and the first and second ultrasound receivers.

15. The method of claim 9 further comprising the steps of:
storing the mechanical strength and structural integrity of the bone;
repeating steps (a) through (f) over time; and
displaying the mechanical strength structural integrity of the bone over time.

16. The method of claim 9 wherein the angle of at least one of said transducers can vary as a function of soft tissue thickness.

17. The method of claim 9 wherein the transducers have a resonance frequency greater than 1 MHz.

18. A handheld portable ultrasound diagnostic device comprising:
three ultrasound transducers, at least one said transducers being focused and angled to propagate a signal through the soft tissue and along the bone;
means for establishing a fixed reference signal for the ultrasound transducers;
means for receiving the propagated ultrasound signal at another of said transducers;
means for sorting the received ultrasound signal according to a relative propagation delay time by correlating the received ultrasound signal with the fixed reference signal; and
means for determining at least one acoustical parameter from the sorted received ultrasound signal propagated along the bone and the surrounding soft tissue and means for relating the at least one acoustical parameter to the mechanical strength and the structural integrity of the bone.

19. The device of claim 18 wherein the acoustical parameters are selected from the group of flight time, pulse duration, rise time, maximum amplitude, counts and energy.

20. The device of claim 18 wherein the acoustical parameters are selected from a velocity, a propagation energy and a degree dispersion.

21. The device of claim 18 wherein the transducers are contained in a housing.

22. The device of claim 18 wherein the angle of at least one of said transducers can vary as a function of soft tissue thickness.

23. The device of claim 18 wherein the transducers have a resonance frequency greater than 1 MHz.
